(19) United States
(12) Patent Application Publication
(10) Pub. No.: US 2025/0235203 A1
(43) Pub. Date: Jul. 24, 2025

(54) SUTURE LOCKING DEVICES FOR PERFORMING TENSIONABLE KNOTLESS SURGICAL PROCEDURES

(71) Applicant: ARTHREX, INC., Naples, FL (US)

(72) Inventors: Andrew Christian PETRY, Naples, FL (US); Peter J. DREYFUSS, Naples, FL (US); Erik KUBIAK, Las Vegas, NV (US)

(21) Appl. No.: 18/421,160

(22) Filed: Jan. 24, 2024

Related U.S. Application Data
(60) Provisional application No. 63/622,989, filed on Jan. 19, 2024.

(51) Int. Cl. A61B 17/04; A61B 17/00; A61B 17/88

(52) U.S. Cl. CPC A61B 17/0487; A61B 17/8869; A61B 2017/00982

(57) ABSTRACT

Systems and methods are provided for performing tensionable knotless surgical procedures. A locking ferrule that includes a one-way locking mechanism may be utilized for tensioning and locking one or more strands of suture during the surgical procedure. The one-way locking mechanism may be established by one or more locking barbs of the suture locking device.

### CROSS-REFERENCE TO RELATED APPLICATIONS

This application claims the benefit of U.S. Provisional Application No. 63/622,989, which was filed on Jan. 19, 2024 and is incorporated hereby by reference in its entirety.

### BACKGROUND

This disclosure relates to the field of surgery, and more particularly to various surgical locking devices that are capable of locking suture when performing a variety of tensionable knotless surgical procedures.

Repetitive trauma to a joint, such as a knee, ankle, hip, elbow, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to systems and methods for performing tensionable knotless surgical procedures, such as tissue repairs, for example. Locking ferrules that include a one-way locking mechanism may be utilized when performing the tensionable knotless surgical procedures for tensioning and locking one or more strands of suture.

An exemplary bone plate assembly may include, inter alia, a bone plate including an opening, a locking ferrule received within the opening and including a one-way locking mechanism, and a suture received through the locking ferrule and locked relative to the bone plate by the one-way locking mechanism.

An exemplary suture anchor assembly may include, inter alia, a suture anchor including an internal bore, a locking ferrule received within the internal bore and including a one-way locking mechanism, and a suture received through the locking ferrule and locked relative to the suture anchor by the one-way locking mechanism.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary bone plate assembly.

FIG. 2 schematically illustrates locking a suture relative to the bone plate assembly of FIG. 1.

FIG. 3 illustrates a locking ferrule of the bone plate assembly of FIG. 1.

FIG. 4 is a cross-sectional view through section 4-4 of FIG. 3.

FIG. 5 illustrates an exemplary locking ferrule.

FIG. 6 illustrates another exemplary locking ferrule.

FIG. 7 illustrates a suture having a varying thickness.

FIG. 8 illustrates another exemplary bone plate assembly.

FIG. 9 illustrates a surgical method for performing a tissue repair.

FIG. 10 illustrates another exemplary locking ferrule.

FIG. 11 illustrates a suture anchor assembly.

FIG. 12 illustrates another surgical method for performing a tissue repair.

### DETAILED DESCRIPTION

This disclosure relates to systems and methods for performing tensionable knotless surgical procedures. A locking ferrule that includes a one-way locking mechanism may be utilized as part of the tensionable knotless repairs for tensioning and locking one or more strands of suture. These and other features of this disclosure are described in further detail below.

An exemplary bone plate assembly may include, inter alia, a bone plate including an opening, a locking ferrule received within the opening and including a one-way locking mechanism, and a suture received through the locking ferrule and locked relative to the bone plate by the one-way locking mechanism.

In any further embodiment, the one-way locking mechanism is configured to allow the suture to be tensioned in a first direction and prevent the suture from moving in a second direction.

In any further embodiment, the one-way locking mechanism is established by a plurality of locking barbs provided within a cannulation of the locking ferrule.

In any further embodiment, the cannulation extends through an eyelet portion of the locking ferrule.

In any further embodiment, an engagement portion is connected to the eyelet portion.

In any further embodiment, the engagement portion includes a pair of flexible retention legs that are configured to clip the locking ferrule to the bone plate.

In any further embodiment, the locking ferrule includes an engagement portion received through the opening and an eyelet portion that extends to a location outside of the opening.

In any further embodiment, the eyelet portion provides the one-way locking mechanism.

In any further embodiment, the one-way locking mechanism includes a plurality of locking barbs that allow the suture to be tensioned in a first direction and prevent the suture from moving in a second direction.

In any further embodiment, the engagement portion includes a pair of flexible retention legs that are configured to clip the locking ferrule to the bone plate.

In any further embodiment, the suture retains the locking ferrule within the opening.

In any further embodiment, the locking ferrule includes a body that is disk-shaped or cylindrical shaped.

An exemplary suture anchor assembly may include, inter alia, a suture anchor including an internal bore, a locking ferrule received within the internal bore and including a one-way locking mechanism, and a suture received through the locking ferrule and locked relative to the suture anchor by the one-way locking mechanism.

In any further embodiment, the locking ferrule includes an engagement portion received within the internal bore and an eyelet portion that extends to a location outside of the internal bore.

In any further embodiment, the eyelet portion provides the one-way locking mechanism.

In any further embodiment, the one-way locking mechanism includes a plurality of locking barbs that allow the suture to be tensioned in a first direction and prevent the suture from moving in a second direction.

In any further embodiment, each of the plurality of locking barbs includes a pointed tip.

In any further embodiment, the engagement portion includes a pair of flexible retention legs that are configured to clip the locking ferrule to the suture anchor.

In any further embodiment, the flexible retention legs are configured to flex toward one another for inserting the locking ferrule into the internal bore and are further configured to flex away from one another to engage a ledge portion of the internal bore.

In any further embodiment, an anchor body of the suture anchor includes a rigid plastic material or a metallic material.

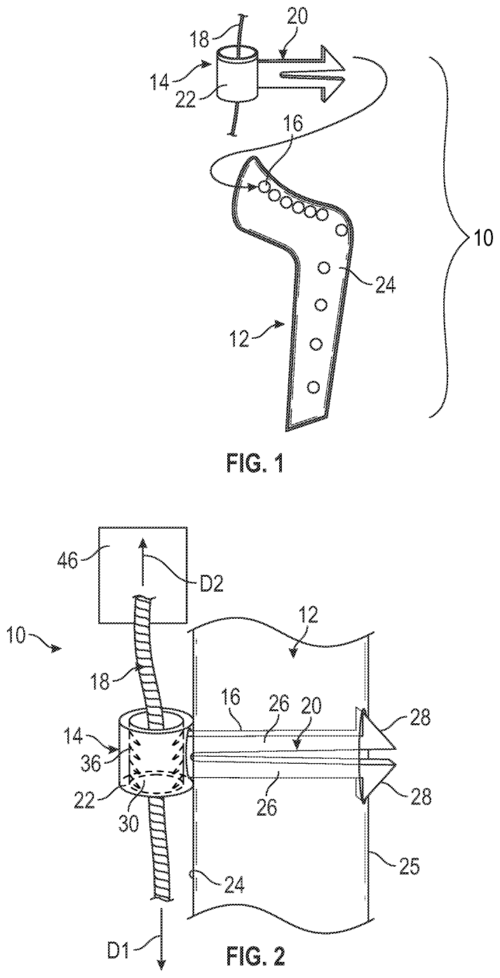

FIGS. 1-2 illustrate an exemplary bone plate assembly 10 that can be used when performing various tensionable knotless surgical procedures. For example, the bone plate assembly 10 could be utilized during surgical methods for performing tissue repairs. The bone plate assembly 10 could be used in conjunction with a variety of orthopedic surgical repairs or reconstructions, including but not limited to cerclage procedures, syndesmosis repairs, clavicle fracture repairs, among many others.

The bone plate assembly 10 is an exemplary suture locking device/system that may include a bone plate 12, one or more locking ferrules 14 that are removably securable relative to the bone plate 12, and one or more sutures 18. The size, shape, and material-makeup of the bone plate 12 are not intended to limit this disclosure.

The bone plate 12 may include one or more openings 16 formed through a plate body of the bone plate 12. The openings 16 may establish through-passages for accommodating other devices, such as suture, screws, etc., and/or for locking one or more strands of suture 18 (see FIG. 2) relative to the bone plate 12.

The locking ferrule 14 may include an engagement portion 20 and an eyelet portion 22. The engagement portion 20 may be received through one of the openings 16 and may be configured to clip to the bone plate 12, for example. When the engagement portion 20 is secured to the bone plate 12, the eyelet portion 22 extends outside of the opening 16 (e.g., above or proud of a top surface 24 of the bone plate 12) and may be configured for receiving and locking the suture 18 relative to the bone plate 12.

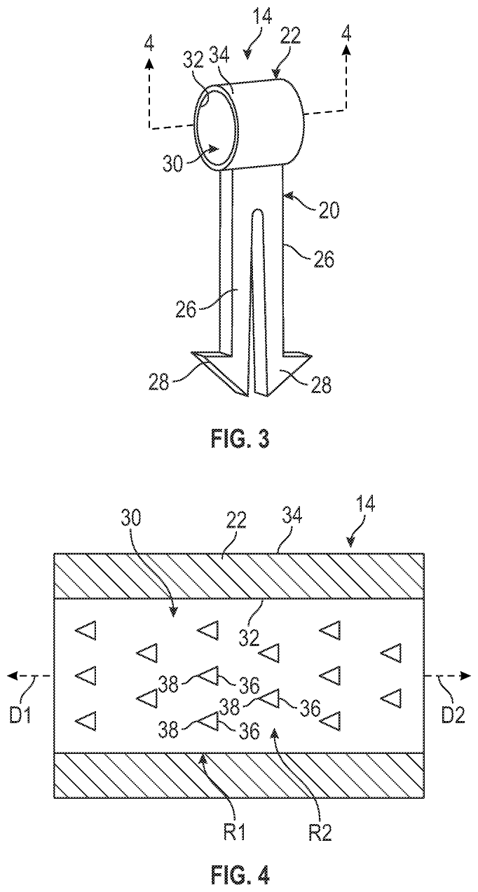

Referring now to FIGS. 3-4, with continued reference to FIGS. 1-2, the engagement portion 20 of the locking ferrule 14 may include a pair of flexible retention legs 26. Each flexible retention leg 26 may include a foot 28 that protrudes outwardly from its respective flexible retention leg 26 at distal end thereof. The flexible retention legs 26 may flex in a first direction (e.g., toward one another) for inserting the locking ferrule 14 through the opening 16 of the bone plate 12, and the flexible retention legs 26 may flex in a second direction (e.g., away from one another) for securing the locking ferrule 14 to the bone plate 12. For example, when the flexible retention legs 26 are flexed in the first direction, a distance between the feet 28 is small enough to pass through the opening 16. Once the feet 28 have been inserted completely through the opening 16, the flexible retention legs 26 may spring outwardly in the second direction to engage a portion (e.g. a bottom surface 25) of the bone plate 12 that surrounds the opening 16, thereby clipping the locking ferrule 14 to the bone plate 12.

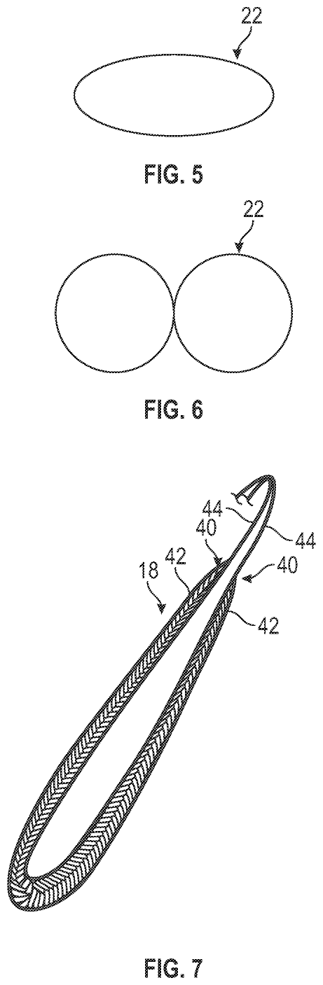

The eyelet portion 22 of the locking ferrule 14 may be either cylindrical shaped (see FIG. 3) or ovoid shaped (see FIG. 5), for example. The eyelet portion 22 may further embody either a single barrel design (see FIG. 3) or a double barrel design (see FIG. 6).

A cannulation 30 may extend through the eyelet portion 22 of the locking ferrule 14. The cannulation 30 may establish a through-passageway for accommodating one or more strands of the suture 18. The cannulation 30 may be circumscribed by an inner diameter wall 32 of the eyelet portion 22. The cannulation 30 may be straight or may taper toward one direction. An outer diameter wall 34 of the eyelet portion 22 may provide a smooth surface.

A plurality of locking barbs 36 may protrude inwardly from the inner diameter wall 32 of the eyelet portion 22. The locking barbs 36 may therefore occupy at least a portion of the open space of the cannulation 30. In an embodiment, the locking barbs 36 are integrally formed (e.g., molded) features of the eyelet portion 22. The locking barbs 36 may be provided along an entire length of the cannulation 30 or at only select portions thereof. The locking barbs 36 may be either rigid or flexible structures.

The locking barbs 36 may be arranged in multiple rows along the length of the cannulation 30. For example, the locking barbs 36 may be arranged in a least a first row R1 and a second row R2 (see FIG. 4). In an embodiment, the locking barbs 36 of the second row R2 are staggered relative to the locking barbs 36 of the first row R1 (see FIG. 4).

Each locking barb 36 may include a sharp or pointed tip 38, and each locking barb 36 may be angled in the same direction, such as toward a common end of the eyelet portion 22. The locking barbs 36 may therefore establish a one-way locking mechanism that permits the suture 18 to pass through the cannulation 30 in a first direction D1 while preventing the suture 18 from being tensioned or otherwise moved in a second direction D2. The staggered relationship of the adjacent rows of locking barbs 36 may provide for maximum engagement with the suture 18 once it has been passed through the cannulation 30.

The suture 18 may be FiberWire®, FiberTape®, or any other suitable suture product. FiberWire® and FiberTape® are suture products marketed and sold by Arthrex, Inc. However, other suture products could be utilized within the scope of this disclosure. The size and type of suture utilized in conjunction with the locking ferrule 14 are not intended to limit this disclosure.

The suture 18 may include a varying thickness. The suture 18 may therefore include one or more tapered regions 40 where the suture 18 transitions between a thickened section 42 and a thinned section 44 (see FIG. 7). The thinned sections 44 can facilitate passing the suture 18 through the cannulation 30, such as via a suitable suture loader that can be provided as part of the suture locking device/system, for example, and the thickened sections 42 can provide greater surface area for the locking barbs 36 to engage in order to sufficiently lock the suture 18 to the locking ferrule 14 and thus relative to the bone plate 12.

In an embodiment, the thickened sections 42 of the suture 18 are about twice as thick as the thinned sections 44. However, other ratios (e.g., 1.5:1, 3:1, etc.) between the relative thicknesses (e.g., outer diameters) of the thickened section 42 and the thinned section 44 are contemplated within the scope of this disclosure.

Either before or after clipping the locking ferrule 14 in place within one of the openings 16 of the bone plate 12, the suture 18 may be passed through or around a tissue 46 (e.g., bone, ligament, tendon, muscle, etc., see FIG. 2) and then be passed through the eyelet portion 22 of the locking ferrule 14. The suture 18 may then be tensioned to knotlessly secure the tissue 46 at a desired position relative to the bone plate 12. Tensioning the suture 18 in the first direction D1 allows the pointed tips 38 of the locking barbs 36 to interdigitate with the portion of the suture 18 accommodated within the cannulation 30, thereby locking the suture 18 in place and preventing it from sliding back in the second direction D2.

Notably, in the above implementations, the cannulation 30 extends along an axis that is substantially parallel to the top surface 24 of the bone plate 12. Accordingly, the suture 18 can be tensioned in a direction that is parallel with the top surface 24 of the bone plate 12. However, the cannulation 30 could alternatively be configured such that the suture 18 is passed and tensioned along a direction that is transverse (e.g., substantially perpendicular) to the top surface 24 of the bone plate 12.

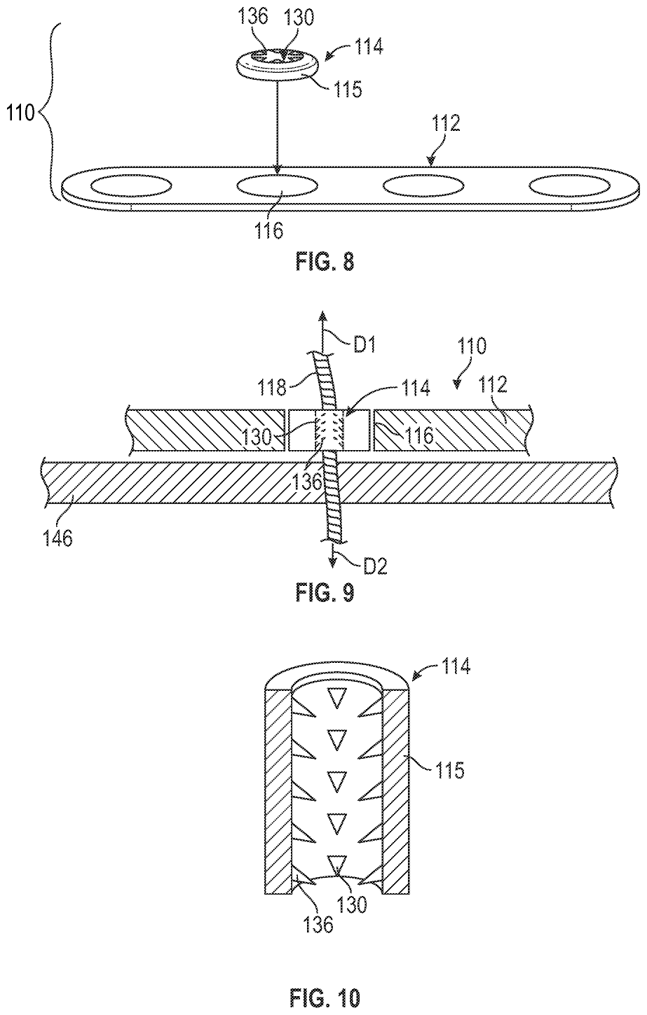

FIGS. 8 and 9 illustrate another exemplary bone plate assembly 110 that may include a bone plate 112, a locking ferrule 114, and suture 118. The bone plate 112 may include one or more openings 116 formed through a plate body of the bone plate 112. The locking ferrule 114 may be positioned within any one of the openings 116 when performing a surgical procedure (e.g., syndesmosis procedure, cerclage procedure, etc.).

The locking ferrule 114 may include a body 115 that can be either disk-shaped (sec FIGS. 8-9) or cylindrical shaped (see FIG. 10), for example. A cannulation 130 may extend through the body 115 for establishing a through-passage for accommodating one or more strands of the suture 118. The cannulation 130 may include a plurality of locking barbs 136. The locking barbs 136 may be arranged in one or more rows along the length of the cannulation 130 and establish a one-way locking mechanism that permits the suture 118 to pass through the cannulation 130 in a first direction D1 while preventing the suture 118 from being tensioned or otherwise moved in a second direction D2.

The suture 118 may be passed through, around or otherwise secured relative to a tissue 146 and may then be passed through the cannulation 130 of the locking ferrule 114. The suture 118 may then be tensioned in the first direction D1 to knotlessly lock the suture 118 relative to the bone plate 112 and thereby secure the tissue 146 at a desired position. In this implementation, the locking ferrule 114 may be retained within the opening 116 of the bone plate 112 by the tension created by locking the suture 118 within the cannulation 130 of the locking ferrule 114 (see, for example, FIG. 9).

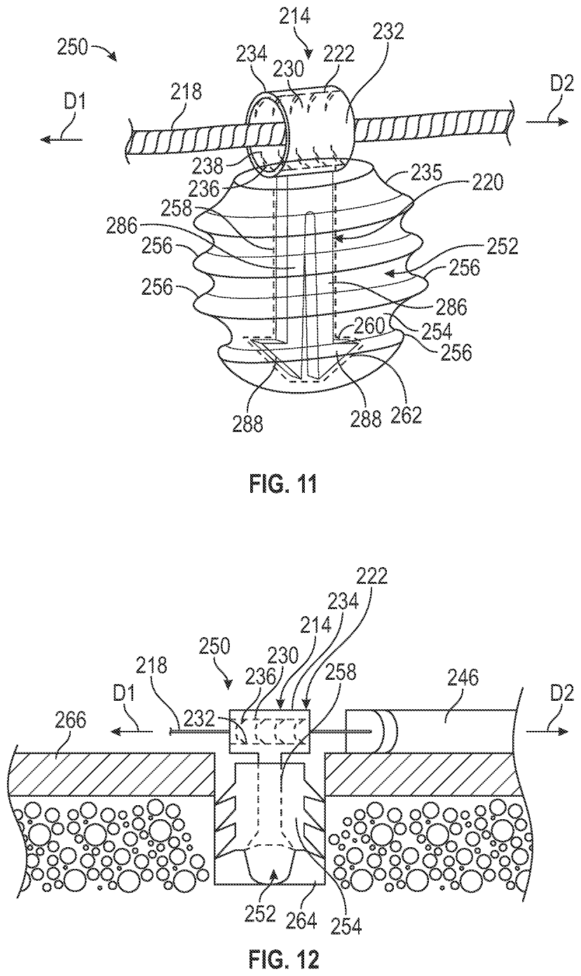

FIGS. 11 and 12 illustrate an exemplary suture anchor assembly 250, which is another suture locking device/system that can be utilized when performing various tensionable knotless surgical procedures (e.g., soft tissue-to-bone fixation, etc.). The suture anchor assembly 250 may include a suture anchor 252, a locking ferrule 214 that may be attached to the suture anchor 252, and a suture 218.

The suture anchor 252 may include an anchor body 254. The anchor body 254 may be a relatively rigid plastic or metallic body. The size and material make-up of the anchor body 254 are not intended to limit this disclosure. An outer wall 235 of the anchor body 254 may include bone engagement features 256 (e.g., threads, barbs, ledges, etc.) for facilitating bone fixation.

The anchor body 254 may include an internal bore 258. The internal bore 258 may longitudinally extend at least partially through the anchor body 254.

The locking ferrule 214 may include an engagement portion 220 and an eyelet portion 222. The engagement portion 220 may be received within the internal bore 258 for attaching the locking ferrule 214 to the anchor body 254. When the engagement portion 220 is secured to the anchor body 254, the eyelet portion 222 is positioned directly over top of the anchor body 254 and may be configured for receiving and locking the suture 218 relative to the suture anchor 252.

The engagement portion 220 of the locking ferrule 214 may include a pair of flexible retention legs 226. Each flexible retention leg 226 may include a foot 228 that protrudes outwardly from its respective flexible retention leg 226 at distal end thereof. The flexible retention legs 226 may flex in a first direction (e.g., toward one another) for inserting the locking ferrule 214 into the internal bore 258 of the suture anchor 252, and the flexible retention legs 226 may flex in a second direction (e.g., away from one another) for securing the locking ferrule 214 to the suture anchor 252. For example, when the flexible retention legs 226 are flexed in the first direction, a distance between the feet 228 is small enough to allow the engagement portion 220 to enter into the internal bore 258. Once the feet 228 have been inserted far enough into the internal bore 258, the flexible retention legs 226 may spring outwardly in the second direction to engage a ledge portion 260 of an expanded diameter section 262 of the internal bore 258, thereby securing the locking ferrule 214 to the suture anchor 252.

A cannulation 230 may extend through the eyelet portion 222 of the locking ferrule 214. The cannulation 230 may establish a through-passage for accommodating one or more strands of suture 218. The cannulation 230 may be circumscribed by an inner diameter wall 232 of the eyelet portion 222. An outer diameter wall 234 of the eyelet portion 222 may provide a smooth surface.

A plurality of locking barbs 236 may protrude inwardly from the inner diameter wall 232 of the eyelet portion 222. The locking barbs 236 may therefore occupy at least a portion of the open space of the cannulation 230. The locking barbs 236 may be arranged in one or more rows along the length of the cannulation 230. Each locking barb 236 may include a sharp or pointed tip 238, and each locking barb 236 may be angled in a common direction relative to the eyelet portion 222. The locking barbs 236 may therefore establish a one-way locking mechanism that permits the suture 218 to pass through the cannulation 30 in a first direction D1 while preventing the suture 218 from being tensioned or otherwise moved in a second direction D2.

In an embodiment, the anchor body 254 of the suture anchor 252 may be inserted into a socket 264 formed in a bone 266. The socket 264 may be a preformed opening formed in the bone 266 that is sized for receiving the anchor body 254 of the suture anchor 252. The suture 218 may be passed through or around a tissue 246 (e.g., ligament, tendon, etc.) and may then be shuttled through the cannulation 230 of the eyelet portion 222 of the locking ferrule 214. The suture 218 may then be tensioned to reduce and knotlessly secure the tissue 246 at a desired position relative to the bone 266.

The suture locking devices/systems of this disclosure may be utilized for performing various tensionable knotless surgical repairs. The suture locking devices/systems provide for tensioning and retensioning suture(s) at various points of the repair, including subsequent to implantation of accompanying fixation devices, thus providing numerous advantages over prior tissue repair systems and techniques.

Notably, the various figures accompanying this disclosure are not necessarily drawn to scale, and some features of the disclosed suture locking systems may be exaggerated or minimized to emphasize certain details of a particular component or system.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A bone plate assembly, comprising:
a bone plate including an opening;
a locking ferrule received within the opening and including a one-way locking mechanism; and
a suture received through the locking ferrule and locked relative to the bone plate by the one-way locking mechanism.

2. The bone plate assembly as recited in claim 1, wherein the one-way locking mechanism is configured to allow the suture to be tensioned in a first direction and prevent the suture from moving in a second direction.

3. The bone plate assembly as recited in claim 2, wherein the one-way locking mechanism is established by a plurality of locking barbs provided within a cannulation of the locking ferrule.

4. The bone plate assembly as recited in claim 3, wherein the cannulation extends through an eyelet portion of the locking ferrule.

5. The bone plate assembly as recited in claim 4, comprising an engagement portion connected to the eyelet portion.

6. The bone plate assembly as recited in claim 5, wherein the engagement portion includes a pair of flexible retention legs that are configured to clip the locking ferrule to the bone plate.

7. The bone plate assembly as recited in claim 1, wherein the locking ferrule includes an engagement portion received through the opening and an eyelet portion that extends to a location outside of the opening.

8. The bone plate assembly as recited in claim 7, wherein the eyelet portion provides the one-way locking mechanism.

9. The bone plate assembly as recited in claim 8, wherein the one-way locking mechanism includes a plurality of locking barbs that allow the suture to be tensioned in a first direction and prevent the suture from moving in a second direction.

10. The bone plate assembly as recited in claim 7, wherein the engagement portion includes a pair of flexible retention legs that are configured to clip the locking ferrule to the bone plate.

11. The bone plate assembly as recited in claim 1, wherein the suture retains the locking ferrule within the opening.

12. The bone plate assembly as recited in claim 11, wherein the locking ferrule includes a body that is disk-shaped or cylindrical shaped.

13. A suture anchor assembly, comprising:
a suture anchor including an internal bore;
a locking ferrule received within the internal bore and including a one-way locking mechanism; and
a suture received through the locking ferrule and locked relative to the suture anchor by the one-way locking mechanism.

14. The suture anchor assembly as recited in claim 13, wherein the locking ferrule includes an engagement portion received within the internal bore and an eyelet portion that extends to a location outside of the internal bore.

15. The suture anchor assembly as recited in claim 14, wherein the eyelet portion provides the one-way locking mechanism.

16. The suture anchor assembly as recited in claim 15, wherein the one-way locking mechanism includes a plurality of locking barbs that allow the suture to be tensioned in a first direction and prevent the suture from moving in a second direction.

17. The suture anchor assembly as recited in claim 16, wherein each of the plurality of locking barbs includes a pointed tip.

18. The suture anchor assembly as recited in claim 14, wherein the engagement portion includes a pair of flexible retention legs that are configured to clip the locking ferrule to the suture anchor.

19. The suture anchor assembly as recited in claim 18, wherein the flexible retention legs are configured to flex toward one another for inserting the locking ferrule into the internal bore and are further configured to flex away from one another to engage a ledge portion of the internal bore.

20. The suture anchor assembly as recited in claim 13, wherein an anchor body of the suture anchor is comprised of a rigid plastic or metallic material.
